(19) United States
(12) Patent Application Publication
(10) Pub. No.: US 2009/0186079 A1
(43) Pub. Date: Jul. 23, 2009

(54) Oral and overall health by negating the biological effects of bacterial lipids

(76) Inventors: Frank C. Nichols, West Hartford, CT (US); Andrei I. Voznesensky, West Hartford, CT (US)

Correspondence Address: ALIX YALE & RISTAS LLP, 750 MAIN STREET, SUITE 1400, HARTFORD, CT, 06103, US

(21) Appl. No.: 12/321,298

(22) Filed: Jan. 20, 2009

Related U.S. Application Data
(60) Provisional application No. 61/011,464, filed on Jan. 17, 2008.

(51) Int. Cl. A61K 9/127; A61K 8/30; A61P 1/02

(52) U.S. Cl. 424/450; 424/49

(57) ABSTRACT

A method of treating an oral cavity condition, a topical composition for use in treating an oral cavity condition and a method of testing a potential agent for its ability to do the same are disclosed. When present in the oral cavity of an individual or animal, bacterial lipids cause various health conditions, such as periodontal disease. Conditions can be treated by any one or more of removal, reduction and/or antagonism of the biological effects of bacterial lipids; solubilizing and removing bacterial lipids; covering, diluting or masking bacterial lipids; antagonizing the cellular signaling of bacterial lipids; and promoting decomposition of bacterial lipids. Conditions can be treated by applying topical compositions containing a safe and effective amount of agents disclosed herein.

### CROSS REFERENCE TO RELATED APPLICATION

This application claims the benefit of U.S. Provisional Application No. 61/011,464, filed Jan. 17, 2008, the disclosure of which is incorporated by reference in its entirety.

### FIELD

This disclosure relates generally to a method for the prophylaxis and treatment of infection by microorganisms in biological environments where the microorganisms deposit lipids. The lipids may remain after the bacteria have been eliminated. In some preferred embodiments this disclosure relates to methods of improving human and animal oral and overall health by limiting or negating biological effects of bacterial lipids.

### BACKGROUND

This disclosure relates generally to methods for treating and preventing diseases, which are accompanied by the deposition of bacterial lipids into the tissues of human or animal host. Periodontal disease is an example of such malady. The disease exists in a number of species of warm-blooded animals such as humans and canines. Periodontal disease is broad term describing the diseases that affect the tissues supporting the teeth. Such tissues are collectively referred to as periodontium. They include the gingiva, periodontal ligament, root cementum, and alveolar bone. Periodontal disease is triggered by the bacterial infections that occur at or below the gum line. In its early form the periodontal disease is limited to surface tissues and is termed gingivitis.

Gingivitis is characterized by inflammation of the gingiva without bone loss or loss of connective tissue attachment. Gingivitis is a common condition, which affects virtually everyone at some point during a lifetime. Gingivitis is graded by severity. Mild gingivitis is characterized by erythema at the sites of inflammation. Moderate gingivitis involves bleeding of the gingiva upon gentle probing, and severe gingivitis is characterized by a tendency for spontaneous gingival bleeding. Gingivitis is a precondition for, but does not necessarily lead to, periodontitis.

Periodontal disease that affects deeper tooth supporting structures is called periodontitis. It can involve all tissues of the periodontium. In periodontitis, oral bacteria accumulate at the junction of the teeth and gingiva causing inflammation of the local periodontal tissues. The inflammation degrades the collagen fibers of the periodontal tissues, causing loss of tooth support and the progressive development of a space between the tooth and the gingiva (periodontal or gingival pocket). As the periodontitis progresses, the periodontal pockets deepen, resulting in inadequate tooth support and tooth loss. Combinations of inflammatory and degenerative conditions are termed periodontitis complex. Other terms used for various aspects of periodontal disease are “juvenile periodontitis”, “acute necrotizing ulcerative gingivitis”, and “alveolar pyorrhea”.

Periodontal disease is the major cause of tooth loss in the adult population. Tooth loss from periodontal disease is a significant problem starting at age 35. Even by age 15 it is estimated that about 4 out of 5 persons already have gingivitis and 4 out of 10 have periodontitis. Brushing the teeth with a cleansing dentifrice may help reduce the incidence of periodontal disease. Unfortunately, good oral hygiene does not necessarily prevent or eliminate periodontal disease because microorganisms contribute to both its initiation and progression. Such traditional treatment may remove, at best, only a small amount of lipid material in the periodontium.

Traditionally treatment of periodontal disease focused on elimination of the pathogenic microorganisms with antibiotic treatment in combination with scaling and root planning. Such traditional treatment may remove, at best, only a small amount of lipid material in the periodontium.

### SUMMARY

Many biological environments are characterized by the presence of microorganisms. Some exemplary biological environments contemplated by the present disclosure include, but are not limited to, dental and bone surfaces, vascular regions and cavities as well as mucosal membranes in animals including mammals, reptiles, amphibians and fish and in avian species as well as hooves of livestock animals. Microorganisms in some biological environments deposit lipids in that environment.

In some cases microorganism presence in the biological environment is deleterious and leads to diseases such as periodontal disease. Traditional therapy to treat the disease seeks to remove the pathogenic microorganisms from the biological environment. However, such traditional therapy may remove, at best, only a small amount of the deposited lipid material. Even after therapy to eliminate pathogenic microorganisms high levels of deposited lipids remain. The presence of such high levels of deposited lipids has been found to interfere with treatment of the disease.

A method of treating an oral cavity condition in a human or animal is disclosed. A safe and effective amount of a bacterial lipid neutralizing agent is administered to the oral cavity. The number of bacteria in the oral cavity is reduced. The amount of bacterial lipids adherent within the oral cavity is also reduced. Tissues within the oral cavity are regenerated.

The bacterial lipid neutralizing agent can comprise at least one of a plurality of materials. Agents can eliminate, reduce or antagonize the biological effects of bacterial lipids. Agents can solubilize and remove bacterial lipids. Agents can cover, dilute or mask bacterial lipids, and thereby negate the biological effects of the lipids. Agents can promote the decomposition of bacterial lipids.

In another embodiment, a topical oral composition for treatment of periodontal disease is disclosed. The composition includes a safe an effective amount of at least one of a plurality of agents. Agents can eliminate, reduce or antagonize the biological effects of bacterial lipids. Agents can solubilize and remove bacterial lipids. Agents can cover, dilute or mask bacterial lipids, and thereby negate the biological effects of the lipids. Agents can promote the decomposition of bacterial lipids. The composition also includes a safe and effective amount of at least one of a plurality of oral therapeutic active materials. Active materials can be antimicrobial agents, antiplaque agents, biofilm inhibiting agents, antibiotics, regeneration promoting agents, enamel matrix protein derivatives, analgesic agents, local anesthetic agents, dentinal desensitizing agents and/or an odor masking agents.

In one embodiment the disclosure, a method of treating a disease lessens the levels of deposited lipids below those achieved in traditional therapy.

In one embodiment, a method of treating a disease in a biological environment is enhanced by lessening the activity of deposited lipids in that environment.

In one embodiment, disease is treated with a bacterial lipid neutralizing agent.

In one embodiment, periodontal disease is treated with compounds that remove, reduce or otherwise antagonize the biological effects of oral microflora lipids.

In one embodiment, a method for discovering a bacterial lipid neutralizing agent to remove, reduce or otherwise antagonize the biological effects of bacterial lipids is disclosed. A bacterial lipid is complexed to a support to form a complexed lipid. The potential agent is contacted with the complexed lipid to form a treated complexed lipid. Cells are exposed to the treated complexed lipid or to the uncomplexed lipid. The effect of the treated complexed lipid on the exposed cells is determined using at least one of a plurality of suitable assays. The results are compared to a control.

### BRIEF DESCRIPTION OF THE DRAWINGS

Referring now to the drawings wherein like elements are numbered alike in the several Figures:

FIG. 1 is a graph illustrating recovery of phosphorylated lipids in bacterial species;

FIG. 2 is a graph illustrating recovery of bacterial lipids from tooth roots;

FIG. 3 is a graph illustrating lipid induced secretion of prostaglandin E2 by peripheral blood monocytes;

FIG. 4 is a series of micrographs illustrating morphological changes induced in peripheral blood monocytes by bacterial lipids;

FIG. 5 is a series of micrographs illustrating morphological changes induced in macrophages by bacterial lipids;

FIG. 6.1 is a series of micrographs illustrating RAW cells exposed to bacterial lipids become TRAP positive;

FIG. 6.2 is a series of micrographs illustrating RAW cells exposed to bacterial lipids become TRAP positive;

FIG. 7 is a series of micrographs illustrating morphological changes induced in osteoblasts by bacterial lipids;

FIG. 8 is a series of micrographs illustrating morphological changes induced in gingival fibroblasts by bacterial lipids;

FIG. 9 is a series of micrographs illustrating a collagen coating shielding gingival fibroblasts from bacterial lipids;

FIG. 10 is a series of micrographs illustrating liposomal treatment negating the biological effect of surface-deposited bacterial lipids; and

FIG. 11 is a series of micrographs illustrating detergent treatment negating the biological effect of surface-deposited bacterial lipids.

### DETAILED DESCRIPTION

For clarity the disclosure will describe the method and materials with respect to an oral biological environment, however the disclosed methods and materials are not limited to the oral biological environment and this disclosure and claims encompass other biological environments.

As used herein “bacterial lipid neutralizing agent” means a drug or agent that can remove, reduce or otherwise antagonize the biological effects of bacterial lipids to a level substantially below the level achievable using traditional treatments such as tooth brushing or scaling and root planning. These agents include, but are not limited to:

agents that solubilize and remove bacterial lipids, such as detergents, phospholipids, buffers, chelating agents, antibodies to bacterial lipids, and combinations thereof; Anionic detergents, including, but not limited to cholic acid, deoxycholic acid, glycocholic acid, taurocholic acid, taurodeoxycholic acid, SDS (sodium dodecyl sulfate); Cationic detergents, including, but not limited to hexadecyltrimethylammonium bromide, tetradecyltrimethylammonium bromide; Zwitterionic detergents, including, but not limited to CHAPS (3-([3-Cholamidopropyl]dimethylammonio)-1-propanesulfonate), CHAPSO (3-[(3-Cholamidopropyl)dimethylammonio]-2-hydroxy-1-propanesulfonate), Zwittergent 3-08 (3-(N,N-Dimethyldodecylammonio)propanesulfonate), Zwittergent 3-14 (3-(N,N-Dimethylmyristylammonio)propanesulfonate), Zwittergent 3-16 (3-(N,N-Dimethylpalmitylammonio)propanesulfonate); Nonionic detergents, including, but not limited to, Triton X-100 (t-Octylphenoxypolyethoxyethanol), Octyl glucoside (N-Octyl beta-D-glucopyranoside), Lauryl-beta-D-maltoside (Dodecyl-beta-D-maltoside), n-Dodecyl beta-D-maltoside, N-Hexadecyl beta-D-maltoside, MEGA-8 (N-Octanoyl-N-methylglucamine), MEGA-9 (N-Nonanoyl-N-methylglucamine), MEGA-10 (N-Decanoyl-N-methylglucamine), Tween 20 (polyoxyethylene(20) sorbitan monolaurate), Tween 40 (polyoxyethylene(20) sorbitan monopalmitate), Tween 60 (polyoxyethylene(20) sorbitan monostearate), Tween 80 (polyoxyethylene(20) sorbitan monooleate), Tween 85 (polyoxyethylene(20) sorbitan trioleate), Brij 35 (polyoxyethylene(23) lauryl ether), Brij 56 (polyoxyethylene(10) cetyl ether)

(a) agents that negate the biological effects of bacterial lipids by covering, diluting and “masking” said lipids such as naturally occurring host lipids, biological detergents, proteins, polypeptides, polymers, and combinations thereof;
(b) agents that serve as functional antagonists of bacterial lipid's cellular signaling such as neutralizing antibodies to bacterial lipids, fragments and functional analogues of said lipids including but not limited to branched fatty acids, polar charged groups, compounds identified through screening of compound libraries and combinations thereof;
(c) agents that promote bacterial lipid decomposition including, but not limited to catalysts, enzymes, microorganisms that decompose bacterial lipids, compounds that stimulate bacterial lipid catabolism by bacteria and/or the host, and combinations thereof.

As used herein “overall health” means overall systemic health characterized by a reduction in risk of development of major systemic diseases including cardiovascular disease, stroke, diabetes, severe respiratory infections, premature and low birth weight infants (including associated post-partum dysfunction in neurologic/developmental function), and associated increased risk of mortality.

As used herein “oral health” means absence of diseases of the oral cavity including periodontal disease, gingivitis, chronic or aggressive periodontitis, periodontitis as a manifestation of systemic disease, periodontosis (outdated term), adult and juvenile periodontitis (outdated terms), and other inflammatory conditions of the tissues within the oral cavity, plus caries, necrotizing ulcerative periodontal disease, resulting conditions from these diseases such as oral or breath malodor, and other conditions such as herpetic lesions, and infections that may develop following dental procedures such as osseous surgery, tooth extraction, periodontal flap surgery, dental implantation, and scaling and root planing. Also oral health would indicate absence of dentoalveolar infections, dental abscesses (e.g., cellulitis of the jaw; osteomyelitis of the jaw), acute necrotizing ulcerative gingivitis (i.e., Vincent's infection), infectious stomatitis (i.e., acute inflammation of the buccal mucosa), and Noma (i.e., gangrenous stomatitis or cancrum oris). Oral and dental infections are more fully disclosed in Finegold, Anaerobic Bacteria in Human Diseases, chapter 4, pp 78-104, and chapter 6, pp 115-154 (Academic Press, Inc., NY, 1977), the disclosures of which are incorporated herein by reference in their entirety. The compositions and methods of treatment of the present disclosure are particularly effective for treating or preventing periodontal disease (gingivitis and/or periodontitis) and resulting breath malodor.

As used herein “topical oral compositions” means a product which in the ordinary course of usage is not intentionally swallowed for purposes of systemic administration of particular therapeutic agents, but is rather retained in the oral cavity for a time sufficient to contact substantially all of the dental surfaces and/or oral tissues for purposes of oral activity.

As used herein “safe and effective amount” means sufficient amount of material to provide the desired benefit (benefit indicating improvement in oral health or healing following periodontal therapy that results in gain of attachment around teeth previously affected by destructive periodontal disease) while being safe to the hard and soft tissues of the oral cavity. The safe and effective amount of bacterial lipid neutralizing agent will vary with the particular condition being treated, the age and physical condition of the patient being treated, the severity of the condition, the duration of treatment, the nature of concurrent therapy, the specific form of the bacterial lipid neutralizing agent employed, and the particular vehicle from which the bacterial lipid neutralizing agent ion is applied.

As used herein the term “carrier” means a suitable vehicle (including excipients and diluents), which is pharmaceutically acceptable and can be used to apply the present compositions in the oral cavity.

As used herein “dentifrice” means toothpaste, tooth powder, and tooth gel formulations unless otherwise specified.

In one preferred method, the disclosed compositions are used to treat and prevent diseases and conditions of the oral cavity including periodontal disease

### Additional Therapeutic Agents

It is recognized that in certain forms of therapy, combinations of therapeutic agents in the same delivery system may be useful in order to obtain an optimal effect. Thus, for example, the compositions may comprise an additional agent such as antimicrobial/antiplaque agents; biofilm inhibiting agents; antibiotics; analgesics and local anesthetic agents; dentinal desensitizing agents; and odor masking agents. The bacterial lipid neutralizing agent may be combined with one or more of such agents in a single delivery system to provide combined effectiveness.

Antimicrobial antiplaque agents may include, but are not limited to, chlorite ion agent; triclosan, 5-chloro-2-(2,4-dichlorophenoxy)-phenol, as described in The Merck Index, 11th ed. (1989), pp. 1529 (entry no. 9573) in U.S. Pat. No. 3,506,720, and in European Patent Application No. 0,251,591 of Beecham Group, PLC, published Jan. 7, 1988; chlorhexidine (Merck Index, no. 2090), alexidine (Merck Index, no. 222); hexetidine (Merck Index, no. 4624); sanguinarine (Merck Index, no. 8320); benzalkonium chloride (Merck Index, no. 1066); salicylanilide (Merck Index, no. 8299); domiphen bromide (Merck Index, no. 3411); cetylpyridinium chloride (CPC) (Merck Index, no. 2024; tetradecylpyridinium chloride (TPC); N-tetradecyl-4-ethylpyridinium chloride (TDEPC); octenidine; delmopinol, octapinol, and other piperidino derivatives; nisin preparations; zinc ion agents; stannous ion agents; essential oils (including thymol, methyl salicylate, eucalyptol, menthol) and analogs and salts of the above antimicrobial antiplaque agents. If present, the antimicrobial antiplaque agents generally comprise at least about 0.01% by weight of the compositions of the present disclosure.

Biofilm inhibiting agents prevent bacterial adherence, colonization in the mouth or maturation into biofilms. Biofilms are defined as bacterial populations adherent to each other and/or to surfaces or interfaces. Biofilm inhibiting agents are thus effective in controlling bacterial populations that mediate periodontal disease and other oral cavity infections. Examples of biofilm inhibiting agents are furanones, cell wall lytic enzymes such as lysozyme, plaque matrix inhibitors such as dextranases and mutanases, and peptides such as bacteriocins, histatins, defensins and cecropins.

Other optional therapeutic agents include antibiotics such as augmentin, amoxicillin, tetracycline, doxycycline, minocycline, metronidazole, neomycin, kanamycin, or clindamycin; dentinal desensitizing agents such as strontium chloride, potassium nitrate, stannous fluoride or sodium fluoride; odor masking agents such as peppermint oil or chlorophyll; local anesthetic agents such as lidocaine or benzocaine; nutritional agents such as amino acids, essential fats, and minerals; and peroxides such as urea peroxide.

### Pharmaceutically-Acceptable Carrier

By “pharmaceutically-acceptable carrier”, as used herein, is meant a suitable vehicle including one or more compatible solid or liquid filler diluents, excipients or encapsulating substances which are suitable for topical, oral administration. By “compatible,” as used herein, is meant that the components of the composition are capable of being commingled without interaction in a manner which would substantially reduce the composition's stability and/or efficacy, according to the compositions and methods of the present disclosure.

The carriers of the present disclosure can include the usual and conventional components of toothpastes (including gels and gels for subgingival application), mouth rinses, mouth sprays, dental solutions including irrigation fluids, chewing gums, and lozenges (including breath mints) as more fully described hereinafter.

The choice of a carrier to be used is basically determined by the way the composition is to be introduced into the oral cavity. If a toothpaste (including tooth gels, etc.) is to be used, then a “toothpaste carrier” is chosen as disclosed in, e.g., U.S. Pat. No. 3,988,433, to Benedict, the disclosure of which is incorporated herein by reference (e.g., abrasive materials, sudsing agents, binders, humectants, flavoring and sweetening agents, etc.). If a mouth rinse is to be used, then a “mouth rinse carrier” is chosen, as disclosed in, e.g., U.S. Pat. No. 3,988,433 to Benedict (e.g., water, flavoring and sweetening agents, etc.). Similarly, if a mouth spray is to be used, then a “mouth spray carrier” is chosen or if a lozenge is to be used, then a “lozenge carrier” is chosen (e.g., a candy base), candy bases being disclosed in, e.g., U.S. Pat. No. 4,083,955, to Grabenstetter et al., which is incorporated herein by reference; if a chewing gum is to be used, then a “chewing gum carrier” is chosen, as disclosed in, e.g., U.S. Pat. No. 4,083,955, to Grabenstetter et al., which is incorporated herein by reference (e.g., gum base, flavoring and sweetening agents). If a sachet is to be used, then a “sachet carrier” is chosen (e.g., sachet bag, flavoring and sweetening agents). If a subgingival gel is to be used (for delivery of actives into the periodontal pockets or around the periodontal pockets), then a “subgingival gel carrier” is chosen as disclosed in, e.g. U.S. Pat. No. 5,198,220, Damani, issued Mar. 30, 1993, P&G, U.S. Pat. No. 5,242,910, Damani, issued Sep. 7, 1993, P&G, all of which are incorporated herein by reference. Carriers suitable for the preparation of compositions of the present disclosure are well known in the art. Their selection will depend on secondary considerations like taste, cost, and shelf stability, etc.

Preferred compositions of the subject disclosure are in the form of dentifrices, such as toothpastes, tooth gels and tooth powders. Components of such toothpaste and tooth gels generally include one or more of a dental abrasive (from about 10% to about 50%), a surfactant (from about 0.5% to about 10%), a thickening agent (from about 0.1% to about 5%), a humectant (from about 10% to about 55%), a flavoring agent (from about 0.04% to about 2%), a sweetening agent (from about 0.1% to about 3%), a coloring agent (from about 0.01% to about 0.5%) and water (from about 2% to about 45%). Such toothpaste or tooth gel may also include one or more of an anticaries agent (from about 0.05% to about 0.3% as fluoride ion), and an anticalculus agent (from about 0.1% to about 13%). Tooth powders, of course, contain substantially all non-liquid components.

Other preferred compositions of the present disclosure are non-abrasive gels, including subgingival gels, which generally include a thickening agent (from about 0.1% to about 20%), a humectant (from about 10% to about 55%), a flavoring agent (from about 0.04% to about 2%), a sweetening agent (from about 0.1% to about 3%), a coloring agent (from about 0.01% to about 0.5%), and the balance water. The compositions may comprise an anticaries agent (from about 0.05% to about 0.3% as fluoride ion), and an anticalculus agent (from about 0.1% to about 13%).

Other preferred compositions of the subject disclosure are mouthwashes, including mouth sprays. Components of such mouthwashes and mouth sprays typically include one or more of water (from about 45% to about 95%), ethanol (from about 0% to about 25%), a humectant (from about 0% to about 50%), a surfactant (from about 0.01% to about 7%), a flavoring agent (from about 0.04% to about 2%), a sweetening agent (from about 0.1% to about 3%), and a coloring agent (from about 0.001% to about 0.5%). Such mouthwashes and mouth sprays may also include one or more of an anticaries agent (from about 0.05% to about 0.3% as fluoride ion), and an anticalculus agent (from about 0.1% to about 3%).

Other preferred compositions of the subject disclosure are dental solutions including irrigation fluids. Components of such dental solutions generally include one or more of water (from about 90% to about 99%), preservative (from about 0.01% to about 0.5%), thickening agent (from 0% to about 5%), flavoring agent (from about 0.04% to about 2%), sweetening agent (from about 0.1% to about 3%), and surfactant (from 0% to about 5%).

Chewing gum compositions typically include one or more of a gum base (from about 50% to about 99%), a flavoring agent (from about 0.4% to about 2%) and a sweetening agent (from about 0.01% to about 20%).

The term “lozenge” as used herein includes: breath mints, troches, pastilles, microcapsules, and fast-dissolving solid forms including freeze dried forms (cakes, wafers, thin films, tablets) and fast-dissolving solid forms including compressed tablets.

The term “fast-dissolving solid form” as used herein means that the solid dosage form dissolves in less than about 60 seconds, preferably less than about 15 seconds, more preferably less than about 5 seconds, after placing the solid dosage form in the oral cavity. Fast-dissolving solid forms are disclosed in copending U.S. patent application Ser. No. 08/253,890, filed Jun. 3, 1994, Brideau; U.S. Pat. No. 4,642,903; U.S. Pat. No. 4,946,684; U.S. Pat. No. 4,305,502; U.S. Pat. No. 4,371,516; U.S. Pat. No. 5,188,825; U.S. Pat. No. 5,215,756; U.S. Pat. No. 5,298,261; U.S. Pat. No. 3,882, 228; U.S. Pat. No. 4,687,662; U.S. Pat. No. 4,642,903. All of these patents are incorporated herein by reference in their entirety.

Lozenges include discoid-shaped solids comprising a therapeutic agent in a flavored base. The base may be a hard sugar candy, glycerinated gelatin or combination of sugar with sufficient mucilage to give it form. These dosage forms are generally described in Remington: The Science and Practice of Pharmacy, 19^{th} Ed., Vol. II, Chapter 92, 1995. Lozenge compositions (compressed tablet type) typically include one or more fillers (compressible sugar), flavoring agents, and lubricants. Microcapsules of the type contemplated herein are disclosed in U.S. Pat. No. 5,370,864, Peterson et al., issued Dec. 6, 1994, which is herein incorporated by reference in its entirety.

In still another aspect, the disclosure comprises a dental implement impregnated with a composition comprising one or more bacterial lipid neutralizing agents. The dental implement comprises an implement for contact with teeth and other tissues in the oral cavity, said implement being impregnated with a safe and therapeutically effective amount of bacterial lipid neutralizing agent. The dental implement can be impregnated fibers including dental floss or tape, chips or strips and polymer fibers. Dental floss or tape typically comprise at least about 0.01 mg bacterial lipid neutralizing agent per cm of material. The dental implement can also be a dental tool used for stimulating the periodontal tissue such as a toothpick or rubber tip.

Types of carriers or oral care excipients which may be included in compositions of the present disclosure, along with specific non-limiting examples, are:

### Abrasives

Dental abrasives useful in the topical, oral carriers of the compositions of the subject disclosure include many different materials. The material selected must be one, which is compatible within the composition of interest and does not excessively abrade dentin. Suitable abrasives include, for example, silicas including gels and precipitates, insoluble sodium polymetaphosphate, hydrated alumina, calcium carbonate, dicalcium orthophosphate dihydrate, calcium pyrophosphate, tricalcium phosphate, calcium polymetaphosphate, and resinous abrasive materials such as particulate condensation products of urea and formaldehyde.

Another class of abrasives for use in the present compositions is the particulate thermo-setting polymerized resins as described in U.S. Pat. No. 3,070,510 issued to Cooley & Grabenstetter on Dec. 25, 1962. Suitable resins include, for example, melamines, phenolics, ureas, melamine-ureas, melamine-formaldehydes, urea-formaldehyde, melamine-urea-formaldehydes, cross-linked epoxides, and cross-linked polyesters. Mixtures of abrasives may also be used.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975, both incorporated herein by reference. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, Zeodent®, particularly the silica carrying the designation Zeodent 119®. The types of silica dental abrasives useful in the toothpastes of the present disclosure are described in more detail in Wason, U.S. Pat. No. 4,340,583, issued Jul. 29, 1982. The abrasive in the toothpaste compositions described herein is generally present at a level of from about 6% to about 70% by weight of the composition. Preferably, toothpastes contain from about 10% to about 50% of abrasive, by weight of the composition.

A particularly preferred precipitated silica is the silica disclosed in U.S. Pat. No. 5,603,920, issued on Feb. 18, 1997; U.S. Pat. No. 5,589,160, issued Dec. 31, 1996; U.S. Pat. No. 5,658,553, issued Aug. 19, 1997; U.S. Pat. No. 5,651,958, issued Jul. 29, 1997, all of which are assigned to the Procter & Gamble Co. All of these patents are incorporated herein by reference in their entirety.

Mixtures of abrasives can be used. All of the above patents regarding dental abrasives are incorporated herein by reference. The total amount of abrasive in dentifrice compositions of the subject disclosure preferably range from about 6% to about 70% by weight; toothpastes preferably contain from about 10% to about 50% of abrasives, by weight of the composition. Solution, mouth spray, mouthwash and non-abrasive gel compositions of the subject disclosure typically contain no abrasive.

### Sudsing Agents (Surfactants)

Suitable sudsing agents are those, which are reasonably stable and form foam throughout a wide pH range. Sudsing agents include nonionic, anionic, amphoteric, cationic, zwitterionic, synthetic detergents, and mixtures thereof. Many suitable nonionic and amphoteric surfactants are disclosed by U.S. Pat. No. 3,988,433 to Benedict; U.S. Pat. No. 4,051,234, issued Sep. 27, 1977, and many suitable nonionic surfactants are disclosed by Agricola et al., U.S. Pat. No. 3,959,458, issued May 25, 1976, both incorporated herein in their entirety by reference.

### a.) Nonionic and Amphoteric Surfactants

Nonionic surfactants which can be used in the compositions of the present disclosure can be broadly defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name Pluronic), polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials.

The amphoteric surfactants useful in the present disclosure can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed.

The present composition can typically comprise a nonionic, amphoteric, or combination of nonionic and amphoteric surfactant each at a level of from about 0.025% to about 5%, preferably from about 0.05% to about 4%, and most preferably from about 0.1% to about 3%.

### b.) Anionic Surfactants

Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. The present composition typically comprises an anionic surfactant at a level of from about 0.025% to about 9%, preferably from about 0.05% to about 7%, and most preferably from about 0.1% to about 5%.

### Fluoride Ions

The present disclosure may also incorporate free fluoride ions. Preferred free fluoride ions can be provided by sodium fluoride, stannous fluoride, indium fluoride, and sodium monofluorophosphate. Sodium fluoride is the most preferred free fluoride ion. Norris et al., U.S. Pat. No. 2,946,725, issued Jul. 26, 1960, and Widder et al., U.S. Pat. No. 3,678,154 issued Jul. 18, 1972, disclose such salts as well as others. These patents are incorporated herein by reference in their entirety.

The present composition may contain from about 50 ppm to about 3500 ppm, and preferably from about 500 ppm to about 3000 ppm of free fluoride ions.

### Thickening Agents

In preparing toothpaste or gels, it is necessary to add some thickening material to provide a desirable consistency of the composition, to provide desirable active agent release characteristics upon use, to provide shelf stability, and to provide stability of the composition, etc. Preferred thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture.

A preferred class of thickening or gelling agents includes a class of homopolymers of acrylic acid crosslinked with an alkyl ether of pentaerythritol or an alkyl ether of sucrose, or carbomers. Carbomers are commercially available from B.F. Goodrich as the Carbopol® series. Particularly preferred carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. No. 5,198,220, Damani, issued Mar. 30, 1993, P&G, U.S. Pat. No. 5,242,910, Damani, issued Sep. 7, 1993, P&G, and U.S. Pat. No. 4,443,430, Mattei, issued Apr. 17, 1984, all of which are incorporated herein by reference.

Thickening agents in an amount from about 0.1% to about 15%, preferably from about 2% to about 10%, more preferably from about 4% to about 8%, by weight of the total toothpaste or gel composition, can be used. Higher concentrations can be used for chewing gums, lozenges (including breath mints), sachets, non-abrasive gels and subgingival gels.

### Humectants

Another optional component of the topical, oral carriers of the compositions of the subject disclosure is a humectant. The humectant serves to keep toothpaste compositions from hardening upon exposure to air, to give compositions a moist feel to the mouth, and, for particular humectants, to impart desirable sweetness of flavor to toothpaste compositions. The humectant, on a pure humectant basis, generally comprises from about 0% to about 70%, preferably from about 5% to about 25%, by weight of the compositions herein. Suitable humectants for use in compositions of the subject disclosure include edible polyhydric alcohols such as glycerin, sorbitol, xylitol, butylene glycol, polyethylene glycol, and propylene glycol, especially sorbitol and glycerin.

### Flavoring and Sweetening Agents

Flavoring agents can also be added to the compositions. Suitable flavoring agents include oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof. Flavoring agents are generally used in the compositions at levels of from about 0.001% to about 5%, by weight of the composition.

Sweetening agents which can be used include sucrose, glucose, saccharin, dextrose, levulose, lactose, mannitol, sorbitol, fructose, maltose, xylitol, saccharin salts, thaumatin, aspartame, D-tryptophan, dihydrochalcones, acesulfame and cyclamate salts, especially sodium cyclamate and sodium saccharin, and mixtures thereof. A composition preferably contains from about 0.1% to about 10% of these agents, preferably from about 0.1% to about 1%, by weight of the composition.

In addition to flavoring and sweetening agents, coolants, salivating agents, warming agents, and numbing agents can be used as optional ingredients in compositions of the present disclosure. These agents are present in the compositions at a level of from about 0.001% to about 10%, preferably from about 0.1% to about 1%, by weight of the composition.

The coolant can be any of a wide variety of materials. Included among such materials are carboxamides, menthol, ketals, diols, and mixtures thereof. Preferred coolants in the present compositions are the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro-and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979; the disclosure of both are herein incorporated by reference in their entirety.

Preferred salivating agents of the present disclosure include Jambu® manufactured by Takasago. Preferred warming agents include capsicum and nicotinate esters, such as benzyl nicotinate. Preferred numbing agents include benzocaine, lidocaine, clove bud oil, and ethanol.

### Anticalculus Agent

The present disclosure also includes an anticalculus agent, preferably a pyrophosphate ion source which is from a pyrophosphate salt. The pyrophosphate salts useful in the present compositions include the dialkali metal pyrophosphate salts, tetraalkali metal pyrophosphate salts, and mixtures thereof. Disodium dihydrogen pyrophosphate (Na₂H₂P₂O₇), tetrasodium pyrophosphate (Na₄P₂O₇), and tetrapotassium pyrophosphate (K₄P₂O₇) in their unhydrated as well as hydrated forms are the preferred species. In compositions of the present disclosure, the pyrophosphate salt may be present in one of three ways: predominately dissolved, predominately undissolved, or a mixture of dissolved and undissolved pyrophosphate.

Compositions comprising predominately dissolved pyrophosphate refer to compositions where at least one pyrophosphate ion source is in an amount sufficient to provide at least about 1.0% free pyrophosphate ions. The amount of free pyrophosphate ions may be from about 1% to about 15%, preferably from about 1.5% to about 10%, and most preferably from about 2% to about 6%. Free pyrophosphate ions may be present in a variety of protonated states depending on a the pH of the composition.

Compositions comprising predominately undissolved pyrophosphate refer to compositions containing no more than about 20% of the total pyrophosphate salt dissolved in the composition, preferably less than about 10% of the total pyrophosphate dissolved in the composition. Tetrasodium pyrophosphate salt is the preferred pyrophosphate salt in these compositions. Tetrasodium pyrophosphate may be the anhydrous salt form or the decahydrate form, or any other species stable in solid form in the dentifrice compositions. The salt is in its solid particle form, which may be its crystalline and/or amorphous state, with the particle size of the salt preferably being small enough to be aesthetically acceptable and readily soluble during use. The amount of pyrophosphate salt useful in making these compositions is any tartar control effective amount, and is generally from about 1.5% to about 15%, preferably from about 2% to about 10%, and most preferably from about 3% to about 8%, by weight of the dentifrice composition.

Compositions may also comprise a mixture of dissolved and undissolved pyrophosphate salts. Any of the above mentioned pyrophosphate salts may be used.

The pyrophosphate salts are described in more detail in Kirk & Othmer, Encyclopedia of Chemical Technology, Third Edition, Volume 17, Wiley-Interscience Publishers (1982), incorporated herein by reference in its entirety, including all references incorporated into Kirk & Othmer.

Optional agents to be used in place of or in combination with the pyrophosphate salt include such known materials as synthetic anionic polymers, including polyacrylates and copolymers of maleic anhydride or acid and methyl vinyl ether (e.g., Gantrez), as described, for example, in U.S. Pat. No. 4,627,977, to Gaffar et al., the disclosure of which is incorporated herein by reference in its entirety; as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof.

### Alkali Metal Bicarbonate Salt

The present disclosure may also include an alkali metal bicarbonate salt. Alkali metal bicarbonate salts are soluble in water and unless stabilized, tend to release carbon dioxide in an aqueous system. Sodium bicarbonate, also known as baking soda, is the preferred alkali metal bicarbonate salt. The present composition may contain from about 0.5% to about 30%, preferably from about 0.5% to about 15%, and most preferably from about 0.5% to about 5% of an alkali metal bicarbonate salt.

### Miscellaneous Carriers

Water employed in the preparation of commercially suitable oral compositions should preferably be of low ion content and free of organic impurities. Water generally comprises from about 5% to about 70%, and preferably from about 20% to about 50%, by weight of the composition herein. These amounts of water include the free water which is added plus that which is introduced with other materials, such as with sorbitol.

Titanium dioxide may also be added to the present composition. Titanium dioxide is a white powder which adds opacity to the compositions. Titanium dioxide generally comprises from about 0.25% to about 5% by weight of the dentifrice compositions.

Other optional agents include synthetic anionic polymeric polycarboxylates being employed in the form of their free acids or partially or preferably fully neutralized water soluble alkali metal (e.g. potassium and preferably sodium) or ammonium salts and are disclosed in U.S. Pat. No. 4,152,420 to Gaffar, U.S. Pat. No. 3,956,480 to Dichter et al., U.S. Pat. No. 4,138,477 to Gaffar, U.S. Pat. No. 4,183,914 to Gaffar et al., and U.S. Pat. No. 4,906,456 to Gaffar et al. Preferred are 1:4 to 4:1 copolymers of maleic anhydride or acid with another polymerizable ethylenically unsaturated monomer, preferably methyl vinyl ether (methoxyethylene) having a molecular weight (M.W.) of about 30,000 to about 1,000,000. These copolymers are available for example as Gantrez (AN 139 (M.W. 500,000), A.N. 119 (M.W. 250,000) and preferably S-97 Pharmaceutical Grade (M.W. 70,000), of GAF Corporation.

### Composition Use

A safe and effective amount of the compositions of the present disclosure comprising a bacterial lipid neutralizing agent may be topically applied to the mucosal tissue of the oral cavity, to the gingival tissue of the oral cavity, and/or to the surface of the teeth, for the treatment or prevention of the above mentioned diseases or conditions of the oral cavity, in several conventional ways. For example, the gingival or mucosal tissue may be rinsed with a solution (e.g., mouth rinse, mouth spray) containing the bacterial lipid neutralizing agent; or if the bacterial lipid neutralizing agent is included in a dentifrice (e.g., toothpaste, tooth gel or tooth powder), the gingival/mucosal tissue or teeth is bathed in the liquid and/or lather generated by brushing the teeth. Other non-limiting examples include applying a non-abrasive gel or paste, which contains the bacterial lipid neutralizing agent, directly to the gingival/mucosal tissue or to the teeth with or without an oral care appliance; chewing gum that contains a bacterial lipid neutralizing agent; chewing or sucking on a breath tablet or lozenge which contains a bacterial lipid neutralizing agent. Preferred methods of applying the bacterial lipid neutralizing agent to the gingival/mucosal tissue and/or the teeth are via rinsing with a mouth rinse solution and via brushing with a dentifrice. Other methods of topically applying the bacterial lipid neutralizing agent to the gingival/mucosal tissue and the surfaces of the teeth are apparent to those skilled in the art.

The concentration of bacterial lipid neutralizing agent in the composition of the present disclosure depends on the type of composition (e.g., toothpaste, mouth rinse, lozenge, gum, etc.) used to apply the bacterial lipid neutralizing agent to the gingival/mucosal tissue and/or the teeth, due to differences in efficiency of the compositions contacting the tissue and teeth, and due also to the amount of the composition generally used. The concentration may also depend on the disease or condition being treated.

For the method of promoting whole body health by treating and preventing diseases or conditions of the oral cavity, including breath malodor, of the present disclosure, a safe and effective amount of the active bacterial lipid neutralizing agent is preferably applied to the gingival/mucosal tissue and/or the teeth (for example, by rinsing with a mouthrinse, directly applying a non-abrasive gel with or without a device, applying a dentifrice or a tooth gel with a toothbrush, sucking or chewing a lozenge or breathmint, etc.) preferably for at least about 10 seconds, preferably from about 20 seconds to about 10 minutes, more preferably from about 30 seconds to about 60 seconds. The method often involves expectoration of most of the composition following such contact. The frequency of such contact is preferably from about once per week to about four times per day, more preferably from about thrice per week to about three times per day, even more preferably from about once per day to about twice per day. The period of such treatment typically ranges from about one day to a lifetime. For particular oral care diseases or conditions the duration of treatment depends on the severity of the oral disease or condition being treated, the particular delivery form utilized and the patient's response to treatment.

If delivery to the periodontal pockets is desirable, such as with the treatment of periodontal disease, a mouthrinse can be delivered to the periodontal pocket using a syringe or water injection device. These devices are known to one skilled in the art. Devices of this type include “Water Pik” by Teledyne Corporation. After irrigating, the subject can swish the rinse in the mouth to also cover the dorsal tongue and other gingival and mucosal surfaces. In addition a toothpaste, non-abrasive gel, toothgel, etc. can be brushed onto the tongue surface and other gingival and mucosal tissues of the oral cavity. The present compositions may also be delivered to tissues and/or spaces within the oral cavity using electromechanical devices such as metering devices, targeted application devices and cleaning or integrated oral hygiene systems. For treating oral tissue wounds and aiding tissue regeneration, fluid subgingival gel compositions that can be inserted via syringe and either a needle or catheter directly into the areas needing treatment, such as the periodontal cavities, are very useful and convenient.

It should be understood that the present disclosure relates not only to methods for delivering the bacterial lipid neutralizing compositions to the oral cavity of a human, but also to methods of delivering these compositions to the oral cavity of other animals, e.g., household pets or other domestic animals, or animals kept in captivity. Other animals include for example, dogs, cats or horses.

Pet care products such as foods, chews and toys are generally formulated to contain from 0.2 mg to 200 mg bacterial lipid neutralizing agent per unit of product to be administered to the animal. The active agent can be incorporated for example, into a relatively supple but strong and durable material such as rawhide, ropes made from natural or synthetic fibers, and polymeric articles made from nylon, polyester or thermoplastic polyurethane. As the animal chews, licks or gnaws the product, incorporated active elements are released into the animal's oral cavity into a salivary medium, comparable to an effective brushing or rinsing. The active agent can be incorporated as an ingredient or ad mixed into a pet food such as for example, a kibbled, semi-moist, or canned food. Highly preferred food embodiments include carriers that tend to increase residence time of the food in the oral cavity. For example, the active agent can be incorporated in a carrier that will tend to stick or adhere to the teeth, in order that a certain amount of product will remain in the mouth and not be ingested immediately. The present compositions may also be incorporated into other pet care products including nutritional supplements and drinking water additives.

The following non-limiting examples further describe preferred embodiments within the scope of the present disclosure. The examples are given solely for illustration and are not to be construed as limitations of this disclosure as many variations thereof are possible without departing from the spirit and scope thereof.

### EXAMPLE 1

### Porphyromonas gingivalis is the Major Producer of Phosphorylated Dihydroceramide Lipids

The lipids of Bacteroides fragilis, Tannerella forsythia, and Prevotella intermedia were extracted as previously described (Nichols, F. C., Riep, B., Mun, J., Morton, M. D., Bojarski, M. T., Dewhirst, F. E. and Smith, M. B. 2004. Structures and biological activity of phosphorylated dihydroceramides of Porphyromonas gingivalis. J Lipid Res. 45:2317-30, Nichols, F. C. 1998. Novel ceramides recovered from Porphyromonas gingivalis: relationship to adult periodontitis. J. Lipid Res. 39:2360-2372).

Briefly, approximately 4 g of lyophilized bacterial pellet was extracted overnight using a modification of the phospholipid extraction procedures of Bligh and Dyer (Can J Biochem Physiol. 1959 August; 37(8): 911-7. A rapid method of total lipid extraction and purification. Bligh E G, Dyer W J) and Garbus et al. (J Biol Chem. 1963 January; 238: 59-63. The rapid incorporation of phosphate into mitochondrial lipids. Garbus J, Deluca H F, Loomans M E, Strong F M.) Specifically, 4 ml of H2O+16 ml of MeOH—CHCl3 (2:1v/v) was added to the bacterial sample and vortexed. After 12 hours, 3 ml of 2 N KCl+0.5 M K2HPO4 and 3 ml CHCl3 were added and the sample vortexed. The lower organic phase was carefully removed and CHCl3 (3 ml) was added to each sample and vortexed. The CHCl3 phase was removed and combined with the previous organic solvent extract. The lipid extract was dried under nitrogen and stored frozen.

The total lipid extracts were evaluated by electrospray-MS. Relative ion abundances are shown for mass spectra with ion masses depicted from 500 to 1000 m/z. The ion abundances for these spectra are based on the total ion abundances acquired from 0-2000 amu and therefore the ion abundances probably closely reflect the relative levels of lipid classes present.

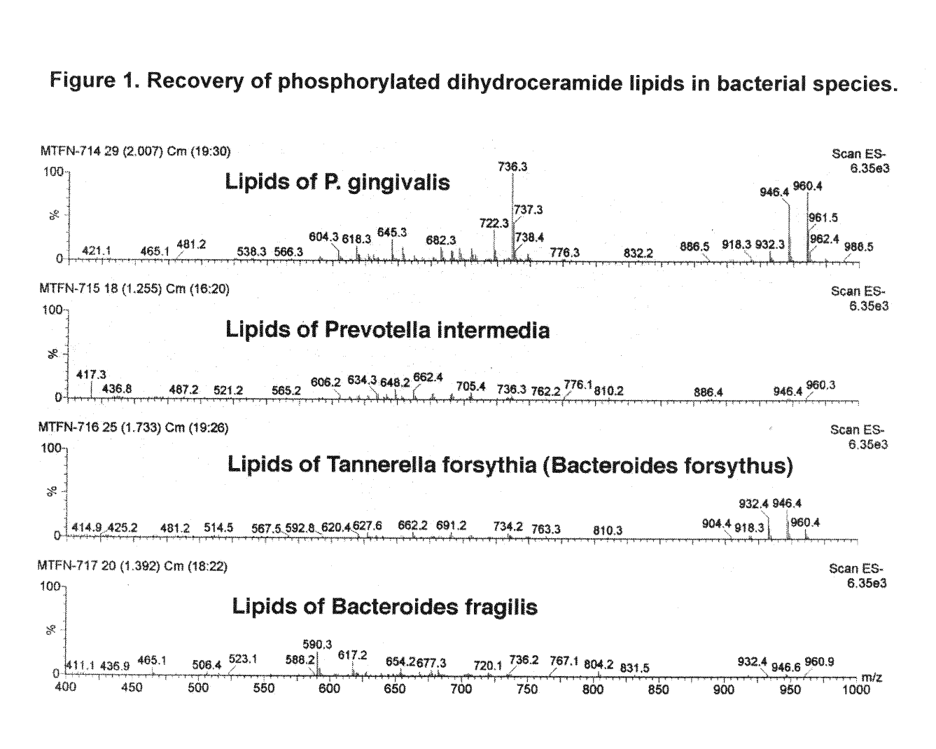

FIG. 1 shows that Porphyromonas gingivalis is the major producer of phosphorylated dihydroceramides followed by Tannerella forsythia. Prevotella intermedia and Bacteroides fragilis produce very low amounts of these lipids. Therefore, Porphyromonas gingivalis is the dominant producer of phosphorylated dihydroceramides.

### EXAMPLE 2

### Bacterial Lipids Strongly Adhere to Dental Root Surfaces

To access the ease of recovery of bacterial lipids from dental root surfaces 10 μg of P. gingivalis lipid in 10 μl of chloroform was applied to root sections from impacted molars. The chloroform was allowed to evaporate and the lipids from the sections were extracted using two methods. Method I used two 1 min washes with 5% phosphoric acid in 95% ethanol:water (7:3, v/v) followed by a 3 hr extraction in chloroform. Method II used two 2 min washes with the same phosphoric acid-ethanol solvent followed by a 3 hr extraction in chloroform.

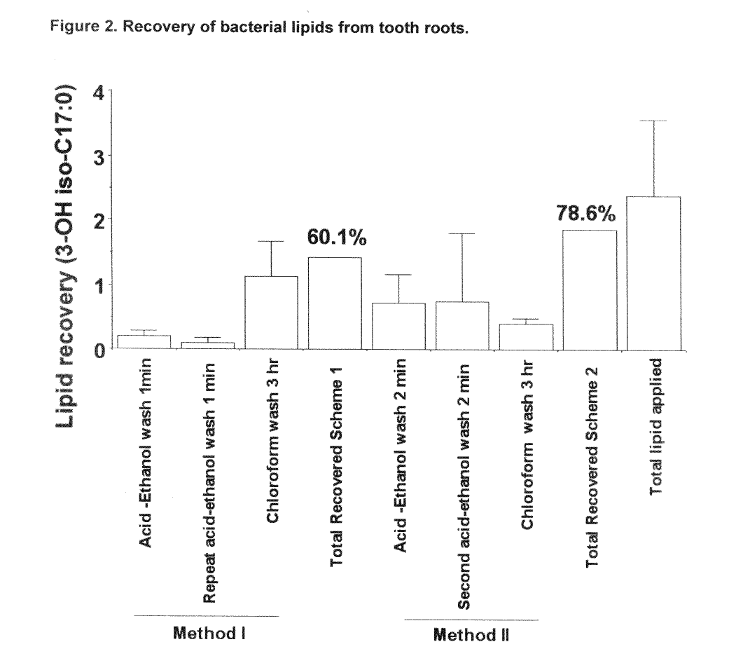

Method I removed approximately 60% of the applied lipids and Method II removed about 78%, as measured by recovery of 3-OH isoC_{17:0} (FIG. 2). The inability of these harsh extraction conditions to remove the lipids indicates that bacterial lipids adhere tightly to dental surfaces.

Also previous reports of bacterial lipid content on cleaned roots most likely significantly underestimated the lipid levels since only a single organic extraction was used (Nichols F C, Levinbook H, Shnaydman M, Goldschmidt J. Prostaglandin E2 secretion from gingival fibroblasts treated with interleukin-1beta: effects of lipid extracts from Porphyromonas gingivalis or calculus. J Periodontal Res. 2001 June; 36(3): 142-52)

This indicates that bacterial lipids strongly adhere to root surfaces and are expected to be found in biologically significant amounts after scaling and root planing.

### EXAMPLE 3

### Surface-Deposited Bacterial Lipids Stimulate Peripheral Blood Monocytes

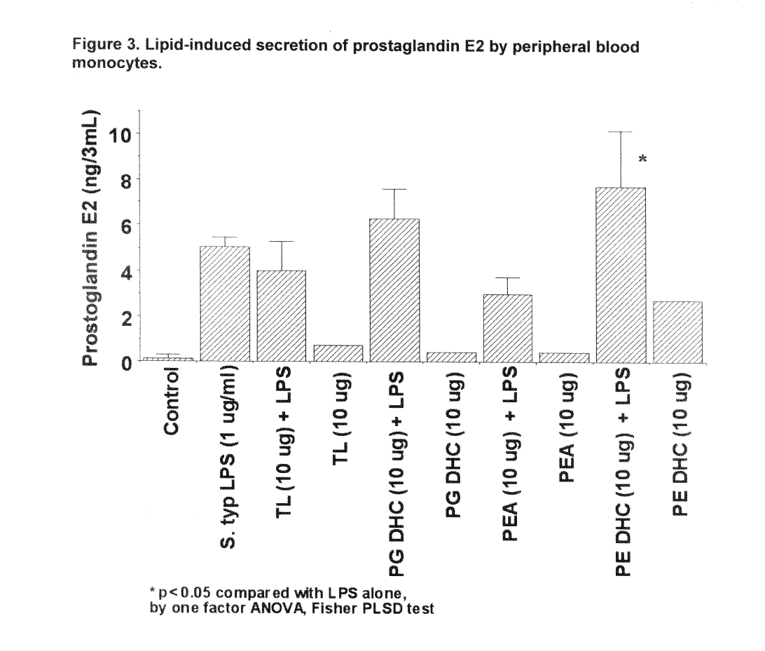

Tissue culture flasks coated with bacterial lipids were used as a model of lipid-coated dental surfaces. Whereas gingival fibroblasts respond most strongly to P. gingivalis phosphoglycerol dihydroceramide lipids (Nichols F C, Riep B, Mun J, Morton M D, Bojarski M T, Dewhirst F E, Smith M B. Structures and biological activity of phosphorylated dihydroceramides of Porphyromonas gingivalis. J Lipid Res. 2004 December; 45(12): 2317-301), LPS-treated monocytes were significantly stimulated by P. gingivalis phosphoethanolamine dihydroceramide lipids (FIG. 3). Peripheral blood monocytes were exposed to lipid film for two hours followed by addition of 1 μg/mL Salmonella typhimurium lipopolysaccharides. Prostoglandin E2 content in the media was measured as previously described (Nichols F C, Riep B, Mun J, Morton M D, Kawai T, Dewhirst F E, Smith M B. Structures and biological activities of novel phosphatidylethanolamine lipids of Porphyromonas gingivalis. J Lipid Res. 2006 April; 47(4): 844-53). TL—total lipid extract of P. gingivalis, LPS—Salmonella typhimurium lipopolysaccharide (1 μg/mL), PG DHC—P. gingivalis phosphoglycerol dihydroceramide lipids, PEA—P. gingivalis phosphoethanolamine dihydroceramide lipids.

As can be seen in FIG. 3, surface-deposited phosphoethanolamine dihydroceramide lipid class alone without LPS increased prostaglandin E₂ secretion substantially over other lipid classes and controls. This indicates that surface deposited bacterial lipids even in the absence of bacterial growth may propagate the periodontal disease.

### EXAMPLE 4

### Surface-Deposited Bacterial Lipids Induce Morphological Changes in Peripheral Blood Monocytes

Tissue culture flasks coated with bacterial lipids were used as a model of lipid-coated biological surfaces. The HPLC-purified P. gingivalis lipids were dissolved in ethanol and added to the tissue culture plate. The solvent was allowed to evaporate in a laminar flow hood overnight leaving the lipid deposited on the culture well surface. Sham-coated control wells received only ethanol solvent.

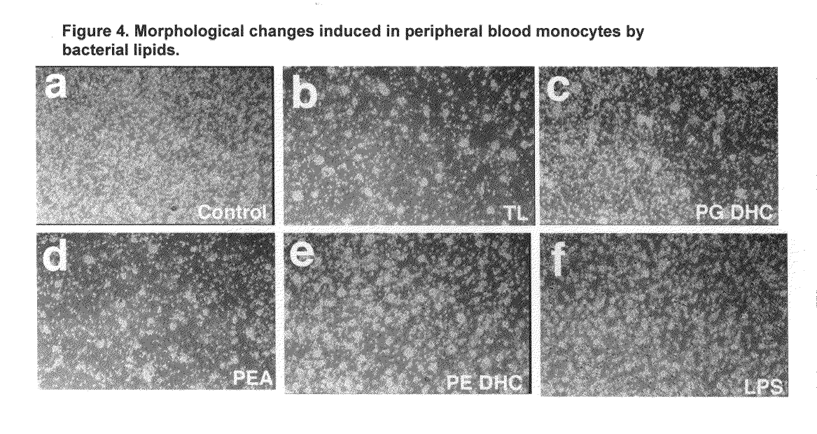

Surprisingly, when peripheral blood monocytes were exposed to surface-deposited P. gingivalis lipid preparations they underwent morphological changes (FIG. 4). Peripheral blood monocytes were plated into 6-well culture dishes previously coated with the indicated lipid fractions (10 μg of lipid/well, 10.5 ng/mm²) and were incubated for 26 hours. Culture (a) was the sham coating control, (b) was coated with the total lipid extract of P. gingivalis, (c) was coated with the P. gingivalis dihydroceramide fraction, (d) was coated with the phosphoethanolamine lipids and (e) was coated with the phosphoethanolamine dihydroceramide lipid fraction, (f) was treated with 1 μg/mL Salmonella typhimurium lipopolysaccharide.

All of the bacterial lipid preparations promote some degree of cell clustering, either through cell migration or division or both. Treatment with lipopolysaccharide did not produce similar cell clustering (Frame f) and co-treatment of monocytes with bacterial lipid preparations plus lipopolysaccharide did not measurably change the appearance of monocytes in culture from the lipid only cultures (pictures not shown). This suggests that lipopolysaccharide do not play a central role in mediating peripheral blood monocyte morphological changes while lipids adhering to the dental surfaces do.

### EXAMPLE 5

### Surface-Deposited Bacterial Lipids Induce Morphological Changes in Macrophages Indicative of Activation

RAW 264.7 cells, a macrophage cell line, were purchased from ATCC (Catalog TIB-71). Cells were maintained in 10 mm dishes in phenol red free DMEM (Sigma-Aldrich Product # D2902) 90% with 10% heat-inactivated FCS, penicillin (100 U/ml) and streptomycin (50 μg/ml). The cells were plated onto lipid-coated 6-well dishes and grown at 37° C. in a humidified atmosphere of 5% CO₂ in air.

Tissue culture flasks coated with bacterial lipids were used as a model of lipid-coated biological surfaces. The HPLC-purified P. gingivalis lipids were dissolved in ethanol and added to the tissue culture plate. The solvent was allowed to evaporate in a laminar flow hood overnight leaving the lipid deposited on the culture well surface. Sham-coated control wells received only ethanol solvent.

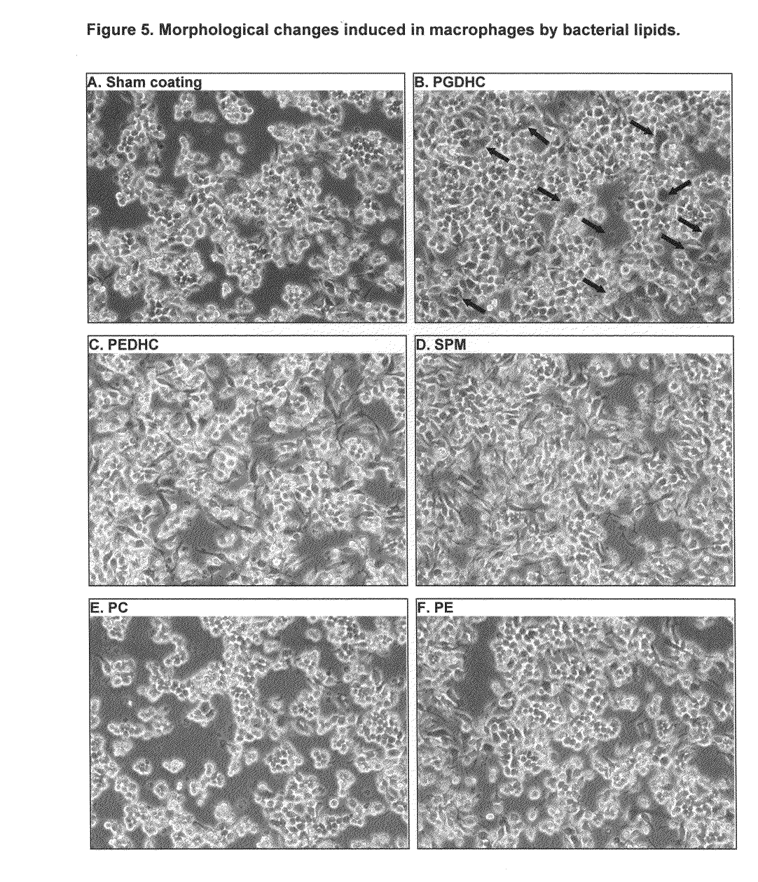

RAW cells were plated into 6-well culture dishes previously coated with the indicated lipid fractions (10 μg of lipid/well, 10.5 ng/mm²) and were incubated for 24 hours. FIG. 5 shows the cultures plated onto wells coated with (A) sham coated control, (B) P. gingivalis phosphoglycerol dihydroceramide, (C) the P. gingivalis phosphoethanolamine dihydroceramide, (D) sphingomyelin, (E) phosphatidylcholine, (F) phosphatidylethanolamine

Surprisingly, when RAW cells were exposed to surface-deposited P. gingivalis phosphoglycerol dihydroceramide they underwent morphological changes suggesting initial stages of osteoclast activation. The cells increased in diameter, and multinucleated cells appeared. Some of the cells with the morphological changes are pointed out by arrows. None of these changes were observed in sham-coated wells (coated with solvent only) or in any of the lipid controls. It is known that activation of macrophages can contribute to the pathogenesis of the periodontal disease.

### EXAMPLE 6

### Surface-Deposited Bacterial Lipids Induce Macrophage Differentiation into Osteoclasts

RAW is a macrophage cell line that potentially can differentiate into osteoclasts, the cells responsible for the breakdown of bone tissue. RAW 264.7 cells were purchased from ATCC (Catalog TIB-71). Cells were maintained in 10 mm dishes in phenol red free DMEM (Sigma-Aldrich Product # D2902) 90% with 10% heat-inactivated FCS, penicillin (100 U/ml) and streptomycin (50 μg/ml). The cells were plated onto lipid-coated 6-well dishes and grown at 37° C. in a humidified atmosphere of 5% CO₂ in air.

Tissue culture flasks coated with bacterial lipids were used as a model of lipid-coated biological surfaces. The HPLC-purified P. gingivalis lipids were dissolved in ethanol and added to the tissue culture plate. The solvent was allowed to evaporate in a laminar flow hood overnight leaving the lipid deposited on the culture well surface. Sham-coated control wells received only ethanol solvent.

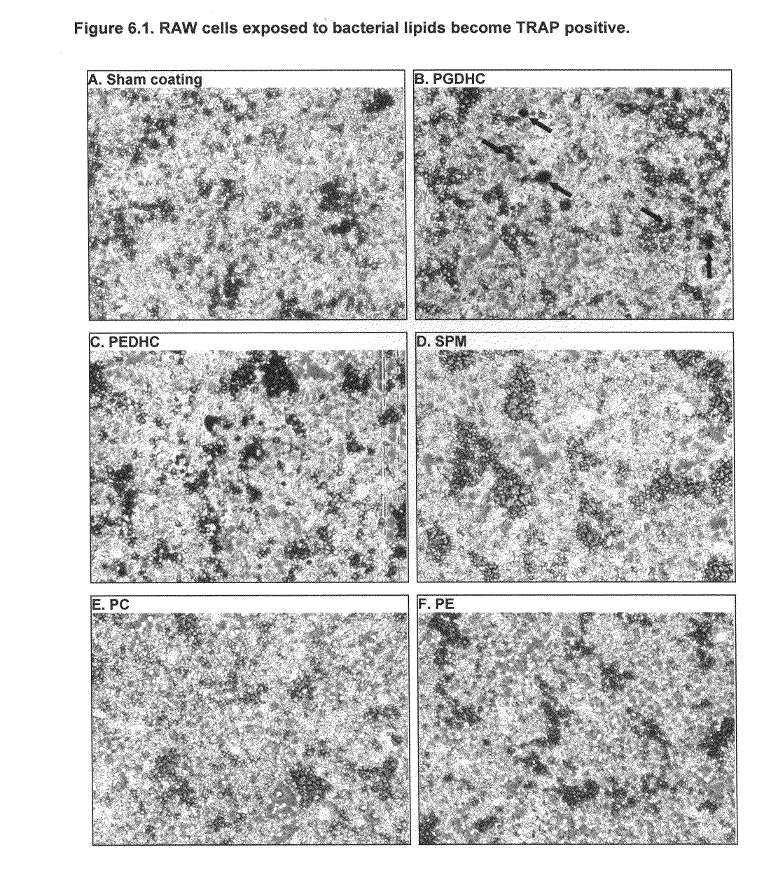

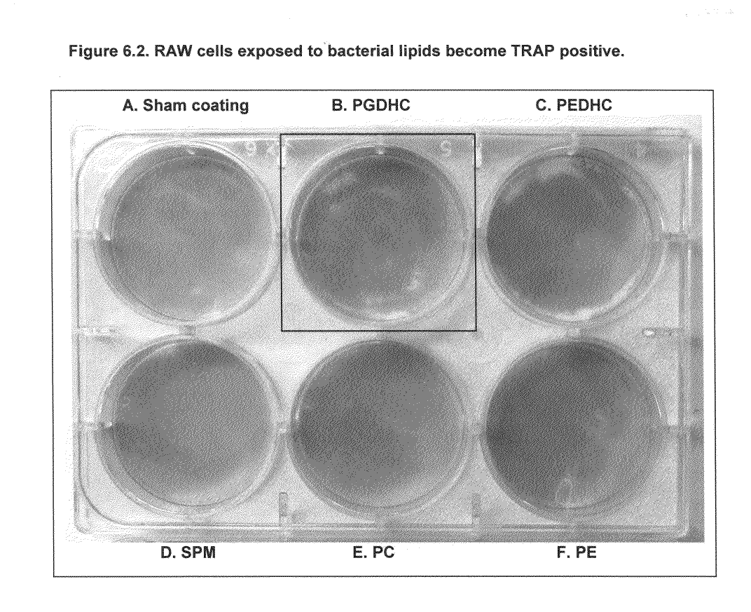

RAW cells were plated into 6-well culture dishes previously coated with the indicated lipid fractions (10 μg of lipid/well, 10.5 ng/mm²). The cells were fixed and stained for Tartrate-Resistant Acid Phosphatase (TRAP) according to Sigma protocol 387. FIG. 6 shows the cultures plated onto wells coated with (A) sham coated control, (B) P. gingivalis phosphoglycerol dihydroceramide, (C) the P. gingivalis phosphoethanolamine dihydroceramide, (D) sphingomyelin, (E) phosphatidylcholine, (F) phosphatidylethanolamine.

Surprisingly, when RAW cells were exposed to surface-deposited P. gingivalis phosphoglycerol dihydroceramide they stained positive for Tartrate-Resistant Acid Phosphatase (TRAP)—a hallmark of activated osteoclasts. Some of the most intensely blue TRAP-positive cells are marked with arrows on FIG. 6.1 in phosphoglycerol dihydroceramide pane. Also note the overall blue TRAP staining of cells in the phosphoglycerol dihydroceramide-coated well in FIG. 6.2. None of these changes were observed in sham-coated wells (coated with solvent only) or in any of the lipid controls. Activation of osteoclast precursors suggests that bacterial lipids deposited on dental surfaces contribute to the etiology of periodontal disease even after the elimination of the microorganism itself. Activation of osteoclasts can lead to bone resorption and manifestation of severe periodontal disease. This indicates that negating the biological effects of bacterial lipids is a viable method of treating periodontal disease.

### EXAMPLE 7

### Surface-Deposited Bacterial Lipids Induce Apoptosis in Preosteoblasts

Osteoblasts are mononucleate cell responsible for bone formation. If bacterial lipids adversely affect osteoblasts it will interfere with the ability of bone to regenerate. Preosteoblast cell line MC3T3-E1 Subclone 4 cells was purchased from ATCC (#CRL-2593). Cells were maintained in 10 mm dishes in Minimum Essential Medium (GIBCO Cat #10370) 90% with 10% heat-inactivated FCS, penicillin (100 U/ml) and streptomycin (50 mg/ml). The cells were plated onto lipid-coated 6-well dishes and grown at 37° C. in a humidified atmosphere of 5% CO2 in air.

Tissue culture flasks coated with bacterial lipids were used as a model of lipid-coated biological surfaces. The HPLC-purified P. gingivalis lipids were dissolved in ethanol and added to the tissue culture plate. The solvent was allowed to evaporate in a laminar flow hood overnight leaving the lipid deposited on the culture well surface. Sham-coated control wells received only ethanol solvent.

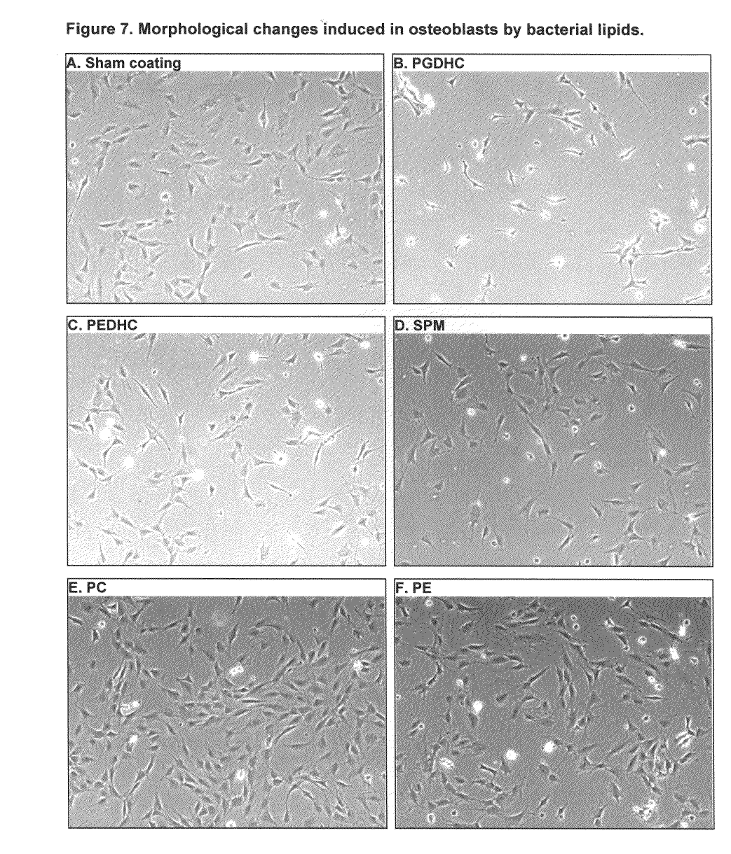

MC3T3-E1 cells were plated into 6-well culture dishes previously coated with the indicated lipid fractions (10 μg of lipid/well, 10.5 ng/mm²) and were incubated for 24 hours. FIG. 7 shows the cultures plated onto wells coated with (A) sham coated control, (B) P. gingivalis phosphoglycerol dihydroceramide, (C) the P. gingivalis phosphoethanolamine dihydroceramide, (D) sphingomyelin, (E) phosphatidylcholine, (F) phosphatidylethanolamine

Surprisingly, when preosteoblast cells were exposed to surface-deposited P. gingivalis phosphoglycerol dihydroceramide they underwent morphological changes suggesting apoptosis (FIG. 7, panel B). Apoptic transformation was confirmed by acridine orange/ethidium bromide staining (data not shown). None of these changes were observed in sham-coated wells (coated with solvent only) or in any of the lipid controls. These data suggest that bacterial lipids deposited on dental surfaces contributes to the pathogenesis of periodontal disease and that removing or antagonizing bacterial lipids is a valid therapeutic strategy.

### EXAMPLE 8

### Surface-Deposited Bacterial Lipids Induce Apoptosis in Fibroblasts

Fibroblasts play a critical part in the elimination of the periodontal pocket. Previously we reported that fibroblasts undergo morphological changes when treated with P. gingivalis lipids. (Nichols F C, Riep B, Mun J, Morton M D, Kawai T, Dewhirst F E, Smith M B. Structures and biological activities of novel phosphatidylethanolamine lipids of Porphyromonas gingivalis. J Lipid Res. 2006 April; 47(4): 844-53, Nichols F C, Riep B, Mun J, Morton M D, Bojarski M T, Dewhirst F E, Smith M B. Structures and biological activity of phosphorylated dihydroceramides of Porphyromonas gingivalis. J Lipid Res. 2004 December; 45(12): 2317-301). Here we show that this effect is specific to phosphoglycerol dihydroceramide.

Human primary gingival fibroblasts were maintained in 10 mm dishes in Minimun Essential Medium (GIBCO Cat #10370) 90% with 10% heat-inactivated FCS, penicillin (100 U/ml) and streptomycin (50 mg/ml). The cells were plated onto lipid-coated 6-well dishes and grown at 37° C. in a humidified atmosphere of 5% CO2 in air. Tissue culture flasks coated with bacterial lipids were used as a model of lipid-coated biological surfaces. The HPLC-purified P. gingivalis lipids were dissolved in ethanol and added to the tissue culture plate. The solvent was allowed to evaporate in a laminar flow hood overnight leaving the lipid deposited on the culture well surface. Sham-coated control wells received only ethanol solvent.

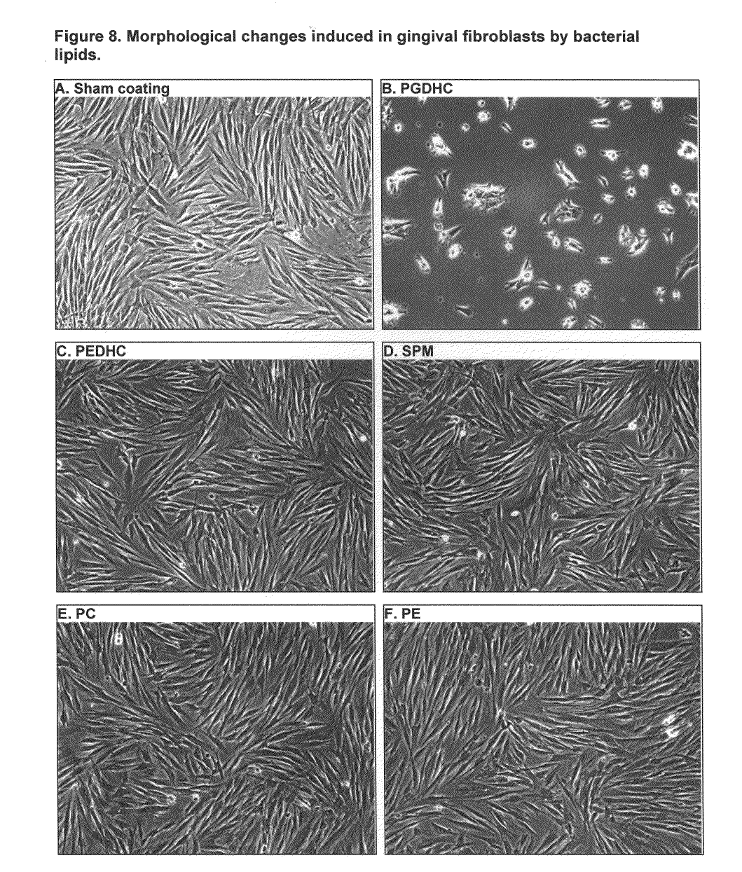

Gingival fibroblasts were plated into 6-well culture dishes previously coated with the indicated lipid fractions (10 μg of lipid/well, 10.5 ng/mm²) and were incubated for 24 hours. FIG. 8 shows the cultures plated onto wells coated with (A) sham coated control, (B) P. gingivalis phosphoglycerol dihydroceramide, (C) the P. gingivalis phosphoethanolamine dihydroceramide, (D) sphingomyelin, (E) phosphatidylcholine, (F) phosphatidylethanolamine

When gingival fibroblasts were exposed to surface-deposited P. gingivalis phosphoglycerol dihydroceramide they underwent morphological changes suggesting apoptosis (FIG. 8, panel B). Apoptic transformation was confirmed by acridine orange/ethidium bromide staining (data not shown). None of these changes were observed in sham-coated wells (coated with solvent only) or in any of the lipid controls. These data suggest that bacterial lipids deposited on dental surfaces contributes to the pathogenesis of periodontal disease and that removing or antagonizing bacterial lipids is a valid therapeutic strategy.

### EXAMPLE 9

### Coating Bacterial Lipid-Bearing Surfaces with Inert Substances can Block Adverse Effects of Bacterial Lipids

Tissue culture flasks coated with bacterial lipids were used as a model of lipid-coated biological surfaces. Human primary gingival fibroblasts were maintained in 10 mm dishes in Minimun Essential Medium (GIBCO Cat #10370) 90% with 10% heat-inactivated FCS, penicillin (100 U/ml) and streptomycin (50 mg/ml). The cells were plated onto the 24-well tissue culture plate with individual wells treated as indicated below and grown at 37° C. in a humidified atmosphere of 5% CO₂ in air.

Lipid coating: The HPLC-purified P. gingivalis phosphoglycerol dihydroceramide was dissolved in ethanol and added to the 24-well tissue culture plate (2 μg of lipid/well, 10.5 ng/mm²). The solvent was allowed to evaporate in a laminar flow hood overnight leaving the lipid deposited on the culture well surface. Sham-coated control wells received only ethanol solvent.

Collagen coating: 0.5 mL of 50 μg/mL collagen dissolved in 0.02N Acetic acid was added to the 24-well tissue culture plate, and incubated at 4° C. overnight on a rocking platform. The collagen solution was aspirated, and the wells were washed with PBS.

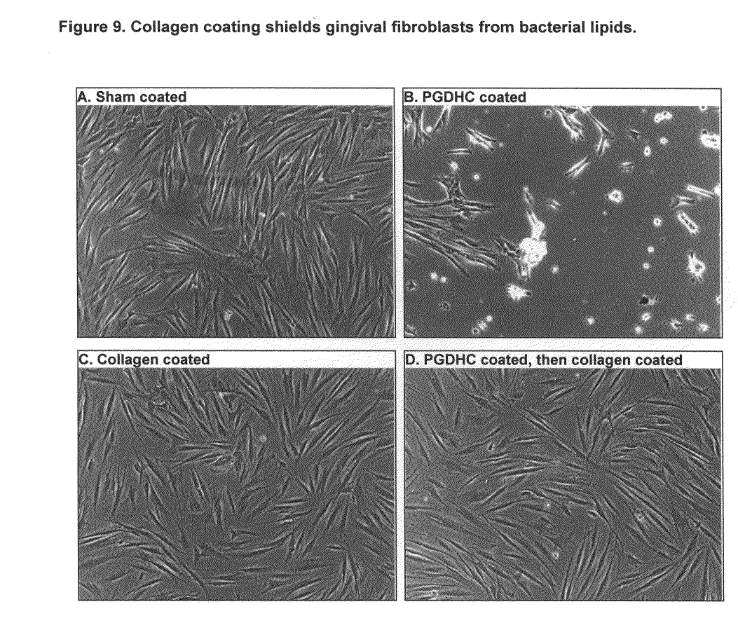

The treatments were done in triplicate. FIG. 9 shows representative pictures of (A) sham-coated control wells, (B) wells coated with P. gingivalis phosphoglycerol dihydroceramide, (C) wells coated with collagen, (D) wells coated with P. gingivalis phosphoglycerol dihydroceramide and then coated with collagen. Fibroblasts depicted in panel A underwent a morphological transformation described in example 8. While collagen coating alone did not have any effect on the primary fibroblasts (panel C), coating the plates with collagen on top of the phosphoglycerol dihydroceramide blocked the biological effect of the P. gingivalis lipid (panel D). The data indicates that the biological effects of bacterial lipids deposited on biological surfaces may be blocked by coating with inert substances, such as collagen.

### EXAMPLE 10

### Treating Bacterial Lipid-Bearing Surfaces with Liposomes Blocks Adverse Effects of Bacterial Lipids

Tissue culture flasks coated with bacterial lipids were used as a model of lipid-coated biological surfaces. Human primary gingival fibroblasts were maintained in 10 mm dishes in Minimun Essential Medium (GIBCO Cat #10370) 90% with 10% heat-inactivated FCS, penicillin (100 U/ml) and streptomycin (50 mg/ml). The cells were plated onto the 6-well tissue culture plate with individual wells treated as indicated below and grown at 37° C. in a humidified atmosphere of 5% CO₂ in air.

Lipid coating: The HPLC-purified P. gingivalis phosphoglycerol dihydroceramide (PGDHC) was dissolved in ethanol and added to the 6-well tissue culture plate (10 μg of lipid/well, 10.5 ng/mm²). The solvent was allowed to evaporate in a laminar flow hood overnight leaving the lipid deposited on the culture well surface. Sham-coated control wells received only ethanol solvent.

Liposomes: Liposomes were prepared by sonicating lipids suspended at 10 mg/mL in phosphate buffered saline (Gibco cat. 1490) in Fisher Scientific Sonic Dismembrator, Model 100 for 3 minutes on a setting of 5. The liposomes were prepared from dimyristoyl L, alpha-phosphatidyl choline (DMPC, Sigma P0888), dilauroyl L, alpha-phosphatidyl ethanolamine (DLPE, Sigma P3642), and dimyristoyl L, alpha-phosphatidyl ethanolamine (DMPE, Sigma P2768). 1 mL of freshly prepared liposomes was added to the wells coated with P. gingivalis phosphoglycerol dihydroceramide. 1 mL of PBS was added to phosphoglycerol dihydroceramide-coated well as a control. The plate was incubated at 4° C. overnight on a rocking platform.

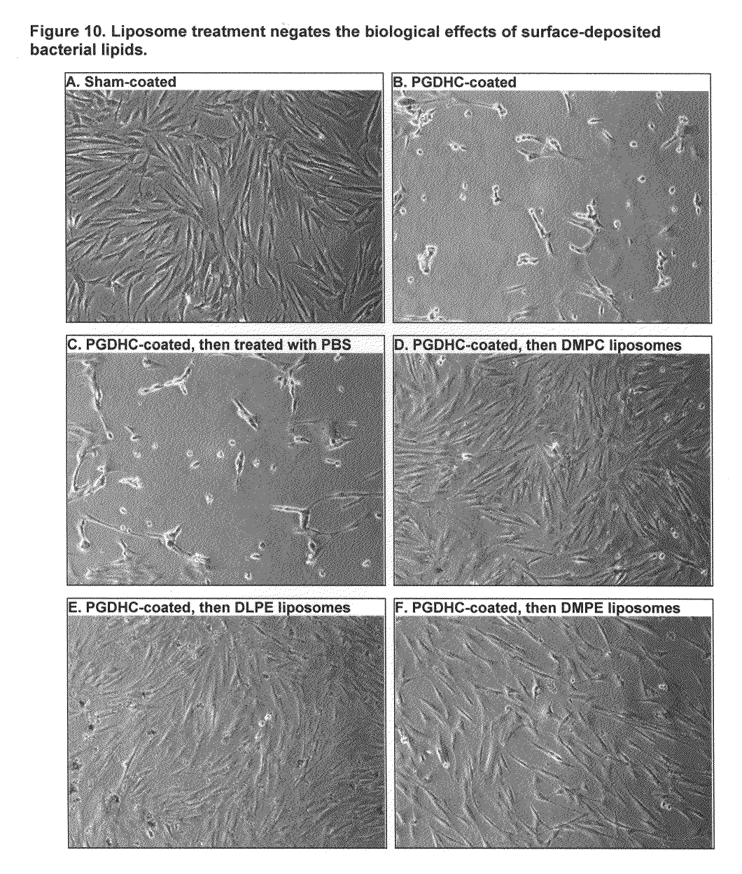

FIG. 10 shows primary gingival fibroblasts on (A) sham-coated surface, (B) surface coated with PGDHC, (C) surface coated with PGDHC, then treated with PBS, (D) surface coated with PGDHC, then treated with DMPC liposomes, (E) surface coated with PGDHC, then treated with DLPE liposomes, (F) surface coated with PGDHC, then treated with DMPE liposomes. Panel B shows morphological transformation of fibroblasts described in example 8. The PGDHC-induced changes could not be reversed by treatment with PBS (panel C), but treatment of the surfaces with either DMPC, DLPE, or DMPE liposomes negated the biological effect of bacterial lipid. Liposomal treatment of uncoated wells had no effect on primary fibroblasts (data not shown). The data indicates that liposomal treatment is viable therapeutic approach to counteracting the effects of bacterial lipids.

### EXAMPLE 11

### Treating Bacterial Lipid-Bearing Surfaces with Detergents Negates the Biological Effects of Bacterial Lipids

Tissue culture flasks coated with bacterial lipids were used as a model of lipid-coated biological surfaces. Human primary gingival fibroblasts were maintained in 10 mm dishes in Minimun Essential Medium (GIBCO Cat #10370) 90% with 10% heat-inactivated FCS, penicillin (100 U/ml) and streptomycin (50 mg/ml). The cells were plated onto the 6-well tissue culture plate with individual wells treated as indicated below and grown at 37° C. in a humidified atmosphere of 5% CO₂ in air.

Lipid coating: The HPLC-purified P. gingivalis phosphoglycerol dihydroceramide (PGDHC) was dissolved in ethanol and added to the 6-well tissue culture plate (10 μg of lipid/well, 10.5 ng/mm²). The solvent was allowed to evaporate in a laminar flow hood overnight leaving the lipid deposited on the culture well surface. Sham-coated control wells received only ethanol solvent.

Detergents: 3-[(3-Cholamidopropyl) dimethylammonio]-1-propanesulfonate (CHAPS), Sodium cholate (Cholate), and Tween-20 (Tween) were dissolved at the concentration of 0.1% in phosphate buffered saline (Gibco cat. 1490). 1 mL of freshly prepared liposomes was added to the wells coated with P. gingivalis phosphoglycerol dihydroceramide. 1 mL of PBS was added to phosphoglycerol dihydroceramide-coated well as a control. The plate was incubated at 4° C. overnight on a rocking platform.

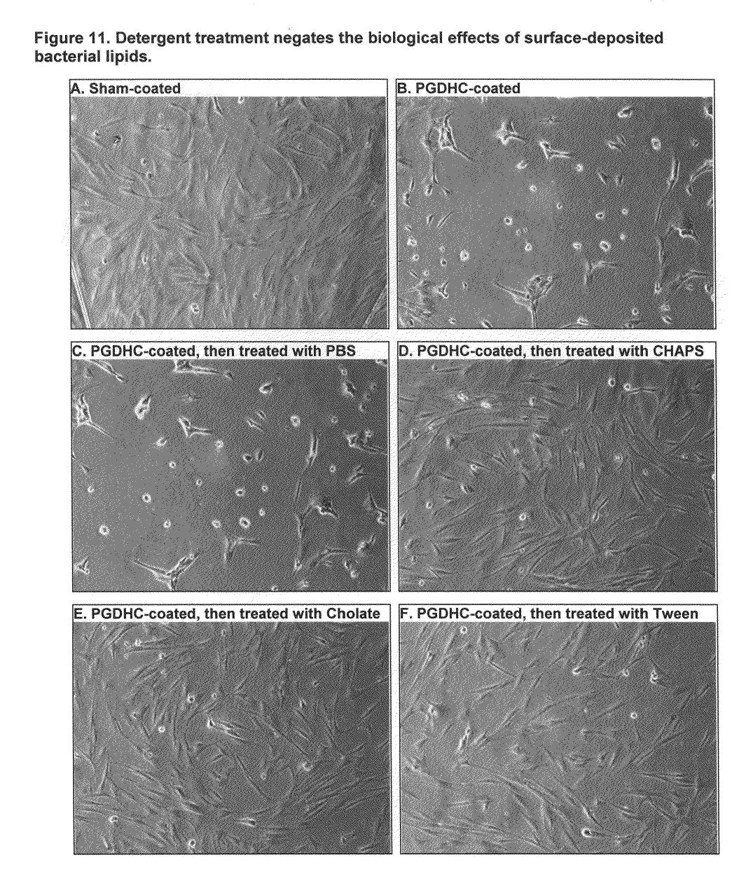

FIG. 11 shows primary gingival fibroblasts on (A) sham-coated surface, (B) surface coated with PGDHC, (C) surface coated with PGDHC, then treated with PBS, (D) surface coated with PGDHC, then treated with 0.1% CHAPS, (E) surface coated with PGDHC, then treated with 0.1% sodium cholate, (F) surface coated with PGDHC, then treated with 0.1% Tween-20. Panel B shows morphological transformation of fibroblasts described in example 8. The PGDHC-induced changes could not be reversed by treatment with PBS (panel C), but treatment of the surfaces with ether zwitterionic, ionic, or non-ionic detergents (CHAPS, sodium cholate, and Tween-20 respectively) negated the biological effect of bacterial lipid. Detergent treatment of uncoated wells had no effect on primary fibroblasts (data not shown). The data indicates that detergent treatment is another viable therapeutic approach to counteracting the effects of bacterial lipids.

1. A method of treating an oral cavity condition in a human or animal in need of such treatment, comprising:
administering a safe and effective amount of a bacterial lipid neutralizing agent to the oral cavity;
reducing the number of bacteria in the oral cavity; and
reducing the amount of bacterial lipids adherent within the oral cavity; and
regenerating tissues within the oral cavity.

2. The method of claim 1 wherein the bacterial lipid neutralizing agent comprises at least one material selected from:
an agent that eliminates, reduces or antagonizes the biological effects of bacterial lipids;
an agent that solubilizes and removes bacterial lipids;
an agent that negates the biological effects of bacterial lipids by covering, diluting or masking the lipids;
an agent that serves as a functional antagonist of the bacterial lipid's cellular signaling; and
an agent that promotes bacterial lipid decomposition.

3. The method of claim 2 wherein the agent that solubilizes and removes bacterial lipids is selected from at least one of a surfactant; a detergent; a phospholipid; a buffer; and a chelating agent.

4. The method of claim 3 wherein the agent that solubilizes and removes bacterial lipids is selected from at least one of a zwitterionic, ionic and non-ionic detergent.

5. The method of claim 3 wherein the agent that solubilizes and removes bacterial lipids is selected from at least one of 3-[(3-Cholamidopropyl) dimethylammonio]-1-propanesulfonate; sodium cholate; and Tween-20.

6. The method of claim 2 wherein the agent that negates the biological effects of bacterial lipids by covering, diluting or masking the lipids is selected from at least one of a naturally occurring host lipid; a biological detergent; a protein; a polypeptide; and a polymer.

7. The method of claim 2 wherein the agent that negates the biological effects of bacterial lipids by covering, diluting or masking the lipids is an inert substance that is coated on a bacterial lipid-containing surface within the oral cavity.

8. The method of claim 7 wherein the inert substance is collagen.

9. The method of claim 2 wherein the agent that negates the biological effects of bacterial lipids by covering, diluting or masking the lipids is at least one type of liposome that is applied to a bacterial lipid-containing surface within the oral cavity.

10. The method of claim 9 wherein the at least one type of liposome is selected from at least one of dimyristoyl L, alpha-phosphatidyl choline (DMPC) liposomes; dilauroyl L, alpha-phosphatidyl ethanolamine (DLPE) liposomes; and dimyristoyl L, alpha-phosphatidyl ethanolamine (DMPE) liposomes.

11. The method of claim 2 wherein the agent that serves as a functional antagonist of the bacterial lipid's cellular signaling is selected from at least one of a branched fatty acid; and a polar charged group.

12. The method of claim 2 wherein the agent that that promotes bacterial lipid decomposition is selected from at least one of a catalyst; an enzyme; a microorganism that decomposes bacterial lipids; a compound that stimulates bacterial lipid catabolism by a bacteria; and a compound that stimulates bacterial lipid catabolism by the human or animal.

13. The method of claim 1 wherein the bacterial lipids are deposited onto a periodontium of the human or animal.

14. The method of claim 1 wherein the bacterial lipids are deposited by oral microflora.

15. The method of claim 1 wherein the oral cavity condition is periodontal disease.

16. The method of claim 1 wherein the step of administering comprises topical application of the bacterial lipid neutralizing agent to at least one of mucosal tissue of the oral cavity; gingival tissue of the oral cavity; and tooth surface.

17. The method of claim 1 wherein the step of administering comprises administering a safe and effective amount of the bacterial lipid neutralizing agent to at least one of a gingival pocket; a periodontal pocket; a tooth root; an alveolar bone; a flap from a periodontal surgical procedure, scaling or root planning; a guided tissue regeneration, an acellular dermal matrix graft, a subepithelial connective tissue graft; and an autogenous free gingival grafts.

18. The method of claim 1 wherein the bacterial lipid neutralizing agent is part of a topical oral composition.

19. A topical oral composition for treatment of periodontal disease, comprising:
a) a safe and effective amount of at least one material selected from:
an agent that eliminates, reduces or antagonizes the biological effects of bacterial lipids;
an agent that solubilizes and removes bacterial lipids;
an agent that negates the biological effects of bacterial lipids by covering, diluting or masking the lipids;
an agent that serves as a functional antagonist of the bacterial lipid's cellular signaling; and
an agent that promotes bacterial lipid decomposition; and
b) a safe and effective amount of an additional oral therapeutic active material selected from an antimicrobial agent; an antiplaque agent; a biofilm inhibiting agent; an antibiotic; a regeneration promoting agent; an enamel matrix protein derivative; an analgesic; a local anesthetic agent; a dentinal desensitizing agent; and an odor masking agent; and
c) a pharmaceutically acceptable oral carrier.

20. A method for screening a potential agent for the ability to remove, reduce or otherwise antagonize the biological effects of bacterial lipids, comprising the steps of:
(a) complexing the lipid to a support to form a complexed lipid;
(b) contacting the potential agent with complexed lipid to form a treated, complexed lipid;
(c) exposing cells to the treated complexed lipid or to uncomplexed lipid;
(d) determining the effect of the treated complexed lipid on the exposed cells using at least one of the following assays: morphological changes; cell proliferation (BrUd, PCNA); apoptosis (TUNEL); p53 mutation accumulation; quantitative and qualitative assessment of cellular lipids; co-clustering patterns of apoptotic and non-apoptotic cell surface receptors; production of pro-inflammatory cytokines; production of immuno-modulatory cytokines; markers of inflammation; anti-apoptotic transcription factors; markers of ageing; cell attachment; and detecting lipid-induced changes in the cell's phosphoprotein profile; and
(e) comparing the results obtained with a control.
